(19) United States
(12) Patent Application Publication
(10) Pub. No.: US 2020/0041437 A1
(43) Pub. Date: Feb. 6, 2020

(54) BIOSENSORS WITH pH-INDEPENDENT REDOX MOIETIES

(71) Applicant: Eccrine Systems, Inc., Cincinnati, OH (US)

(72) Inventors: Jacob A. Bertrand, Norwood, OH (US); Brian Hanley, Cincinnati, OH (US); Michael Brothers, Lebanon, OH (US)

(73) Assignee: Eccrine Systems, Inc., Cincinnati, OH (US)

(21) Appl. No.: 16/339,121

(22) PCT Filed: Oct. 3, 2017
(86) PCT No.: PCT/US17/54875

§ 371 Date: Apr. 3, 2019

Related U.S. Application Data
(60) Provisional application No. 62/403,341, filed on Oct. 3, 2016.

(51) Int. Cl. G01N 27/327; G01N 27/30; A61B 5/145

(52) U.S. Cl. CPC G01N 27/3277; A61B 5/1451; A61B 5/14517; G01N 27/302

(57) ABSTRACT

An electrochemical sensor is provided for use in a wearable device for measuring analytes in a pH-variable biofluid. The sensor includes a plurality of aptamer sensing elements which have biorecognition elements, such as aptamers, that experience a conformational change on interaction with a target analyte in the biofluid. Each aptamer sensing element forms a first configuration before target analyte capture and a second configuration after target analyte capture. A redox moiety is paired with each aptamer sensing element. The redox moiety has a reaction potential that is at least partially independent of a pH value of the biofluid. The EAB sensor further includes an electrode operative in conjunction with the plurality of aptamer sensing elements to produce a variable signal depending upon the configuration of the aptamer sensing elements.

### CROSS-REFERENCE TO RELATED APPLICATIONS

This application claims priority to PCT/US17/54875, filed Oct. 3, 2017, and U.S. Provisional Application No. 62/403,341, filed Oct. 3, 2016, the disclosures of which are hereby incorporated herein by reference in their entirety.

### BACKGROUND OF THE INVENTION

Despite the many ergonomic advantages of sweat compared to other biofluids (particularly in wearable devices), sweat remains an underutilized source of biomarker analytes compared to the established biofluids: blood, urine, and saliva. A number of challenges, however, have historically kept sweat from occupying its place among the preferred clinical biofluids. These challenges include very low sample volumes (nL to μL), unknown concentration due to evaporation, filtration and dilution of large analytes, mixing of old and new sweat, and the potential for contamination from the skin surface. More recently, rapid progress in wearable sweat sampling and sensing devices has resolved several of the historical challenges. However, this recent progress has also been limited to high concentration analytes (μM to mM) sampled at high sweat rates (>1 nL/min/gland) found in, for example athletic applications. Progress will be much more challenging as sweat biosensing moves towards detection of large, low concentration analytes (nM to pM and lower).

Many known sensor technologies for detecting larger molecules are ill-suited for use in wearable sweat sensing, which requires sensors that permit continuous use on a wearer's skin. This means that sensor modalities that require complex microfluidic manipulation, the addition of reagents, the use of limited shelf-life components, such as antibodies, or sensors that are designed for a single use will not be sufficient for sweat sensing. For example, it is known to use ferric cyanide as a redox moiety in an analyte sensor. However, these sensors require the ferric cyanide to be added as a reagent in order to functionalize the sensors. The need to add a reagent prevents these sensors from being useful for continuous use, wearable biosensing applications.

Electrochemical aptamer-based (“EAB”) sensor technology, such as the multiple-capture EAB sensors disclosed in U.S. Pat. Nos. 7,803,542, and 8,003,374, presents a stable, reliable bioelectric sensor that is sensitive to the target analyte in sweat. Similarly, U.S. Provisional Application No. 62/523,835, filed Jun. 23, 2017 and incorporated by reference herein in its entirety, presents a docked aptamer EAB sensor also for use with the disclosed invention.

As disclosed in PCT/US17/23399, filed Mar. 21, 2017, incorporated by reference in its entirety herein, EAB sensors for use in continuous sweat sensing are configured to provide stable sensor responses over time in the presence of a mostly continuous or prolonged flow of sweat sample. For example, the multiple-capture EAB sensor includes a plurality of individual aptamer sensing elements, as depicted in FIG. 1A, which can repeatedly detect the presence of a molecular target by capturing and releasing target analytes as they interact with the aptamer. The sensing element includes an analyte capture complex 140 that has a first end covalently bonded to a first primer 142, and a second end bonded to a second, complementary primer 144. One of the primers, here the first primer 142, is bonded to a sulfur molecule (thiol) 120, which is in turn covalently bonded to a gold electrode base 130. In other embodiments (not shown), the aptamer may be bound to the electrode by means of an ethylenediaminetetraacetic acid (EDTA) strain, to improve adhesion in difficult sensing environments, such as sweat biofluid. The sensing element further includes a redox moiety 150 that may be covalently bonded to the aptamer 140 or bound to it by a linking section. In the absence of the target analyte, the aptamer 140 is in a first configuration, and the redox moiety 150 is in a first position relative to the electrode 130. When the sweat sensing device interrogates the sensing element using square wave voltammetry (SWV), the sensing element produces a first electrical signal, eT_{A}.

With reference to FIG. 1B, the aptamer 140 is selected to specifically interact with a target analyte 160, so that when the aptamer captures a target analyte molecule, the aptamer undergoes a conformation change that partially disrupts the first configuration, and forms a second configuration. In this second configuration, the complementary primers 142, 144 have been brought in close proximity to each other, allowing them to bond. The capture of the target analyte 160 accordingly moves the redox moiety 150 into a second position relative to the electrode 130. Now when the sweat sensing device interrogates the sensing element, the sensing element produces a second electrical signal eT_{B} that is moiety 150 into a second position relative to the electrode 130. Now when the sweat sensing device interrogates the sensing element, the sensing element produces a second electrical signal eT_{B} that is distinguishable from the first electrical signal. After a time interval of nanoseconds, milliseconds, seconds or longer, (the “recovery interval”), the aptamer 140 releases the target analyte 160, and the aptamer returns to the first configuration, which will produce the corresponding first electrical signal when the sensing element is interrogated.

Current state of the art EAB sensors commonly utilize a methylene blue (MB) molecule as a redoxable moiety because its behavior is well understood, it has a suitably low redox reaction potential, and it is stable during typical electrochemical processes. In testing media with very stable and narrow pH ranges, such as blood, MB is typically utilized as the redox moiety due to the consistent performance of the molecule through multiple signal-on/signal-off analyte capture cycles. However, one challenge with the use of EAB sensor technology in sweat applications is that the electrical outputs from such sensors often have a strong dependence on pH. Sweat pH is not stable, and can vary as much as 300×, from about 4.5 to about 7. The pH dependence seen in EAB sensors is primarily due to the effect pH has on the reactive potential of the redox moiety that produces the electrical signal indicating analyte capture. Methylene blue's redox potential depends both on its protonation state and, as depicted in FIG. 2, its reliance upon a proton (H⁺) transfer to perform the redox reaction. The free amines on the ring system of MB can react with acidic protons and change the electron transfer energy enough to affect the signal, thereby causing high variability in the sensor signal across different pH.

One solution to mitigate the effect of pH variability on the sensor signal would be to add a pH sensor, and use the sensor readings to correct for pH-induced errors in the EAB output signal. For example, integration of a pH sensor in a sweat biosensing device is disclosed in PCT/US15/40113, incorporated herein by reference in its entirety. Another solution would be to buffer the biofluid sample for pH, as disclosed in PCT/US16/58357, incorporated herein by reference in its entirety. However, due to the additional complexity of adding sensors to correct for pH variability, or buffering biofluid sample pH, these techniques are less desirable than reducing the pH sensitivity of the EAB sensor. Accordingly, it is desirable to have a pH independent EAB sensor, as well as simple, yet robust, methods to reduce the effect of biofluid pH variability on the output of one or more redox mediated sensors in an EAB sensing device.

### SUMMARY OF THE INVENTION

Devices and methods are described herein for mitigating the effect of pH variability in signals from biofluid sensing devices using redox-mediated aptamer sensors. An aptamer sensing element is functionalized on an electrode and capable of detecting one or more analytes in a pH-variable biofluid sample. The sensing element includes a redox moiety having a redox reaction potential that is at least partially independent of a pH value of the sample. An analyte capture complex is paired with the redox moiety. The analyte capture complex experiences a conformation change on capture of a target analyte in the sample. The complex forms a first configuration relative to the electrode before target analyte capture, and a second configuration relative to the electrode after target analyte capture. The conformation change produces a detectable signal change upon interrogation of the electrode.

### BRIEF DESCRIPTION OF THE DRAWINGS

The objects and advantages of the disclosed invention will be further appreciated in light of the following detailed description and drawings in which:

FIGS. 1A and 1B are schematic representations of a previously-disclosed EAB sensing element;

FIG. 2 is a depiction of a methylene blue redox reaction;

FIG. 3 is a depiction of a viologen redox reaction;

FIG. 4 depicts a methyl viologen redox moiety;

FIGS. 5A to 5F is a depiction of a chemical process to create a methyl viologen redox moiety;

FIG. 6 is a depiction of a hexyl methyl viologen redox reaction;

FIGS. 7A to 7E is a depiction of a chemical process to create a hexyl methyl viologen redox moiety;

FIG. 8 is a depiction of an alternative embodiment of a pH-independent redox moiety for use in an EAB sensor;

FIG. 9 is a depiction of an alternative embodiment of a pH-independent redox moiety useable in an EAB sensor;

FIGS. 10A through 10E depict additional embodiments of nitrogen containing ring systems useable as a redox moiety in an EAB sensor;

FIG. 11 is a depiction of an alternative embodiment of a pH-independent redox moiety useable in an EAB sensor;

FIG. 12 is a depiction of an alternative embodiment of a pH-independent redox moiety useable in an EAB sensor; and

FIGS. 13A and 13B depict an alternative configuration for an aptamer sensing element.

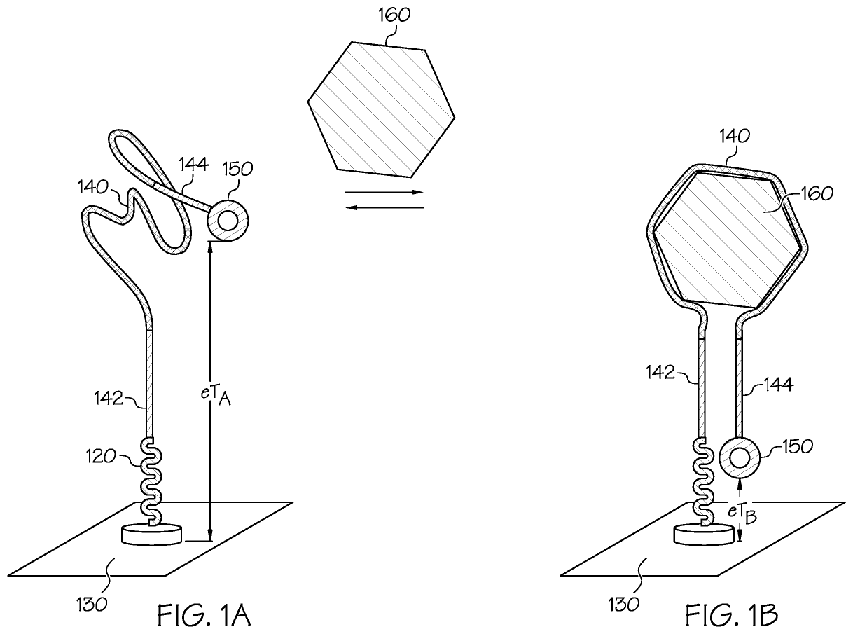

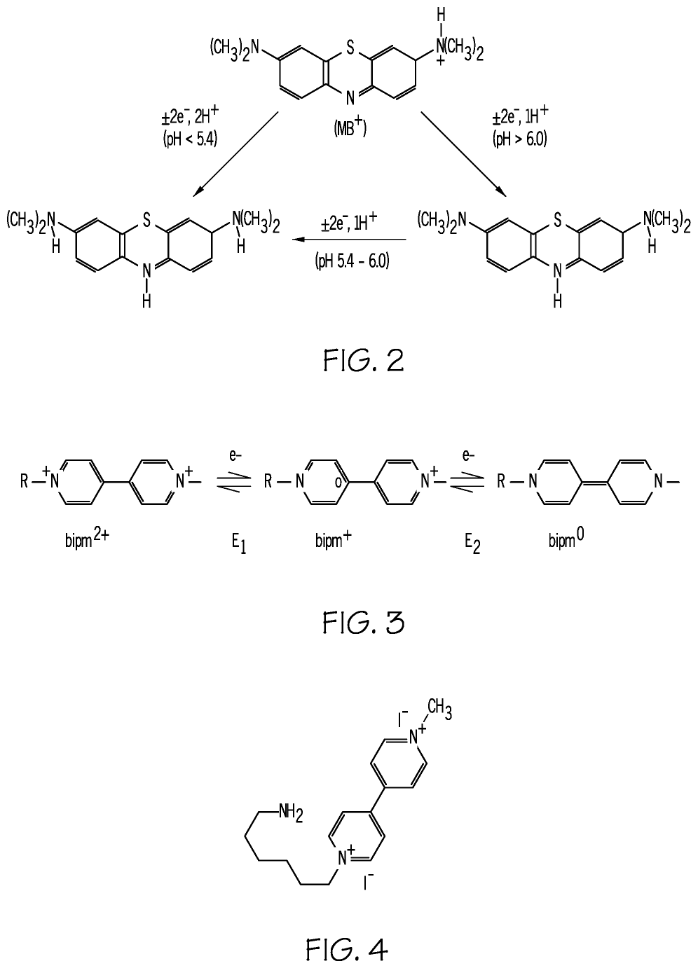

### DEFINITIONS

As used herein, “continuous monitoring” means the capability of a device to provide at least one measurement of sweat determined by a continuous or multiple collection and sensing of that measurement or to provide a plurality of measurements of sweat over time.

As used herein, “interstitial fluid” is a solution that bathes and surrounds tissue cells. The interstitial fluid is found in the interstices or spaces between cells. Embodiments of the disclosed invention measure analytes from interstitial fluid found in the skin and, particularly, interstitial fluid found in the dermis. In some cases where interstitial fluid is emerging from sweat ducts, the interstitial fluid contains some sweat as well, or alternately, sweat may contain some interstitial fluid.

As used herein, “biofluid” may mean any human biofluid, including, without limitation, sweat, interstitial fluid, blood, plasma, serum, tears, and saliva. For sweat sensing applications as generally discussed herein, biofluid has a narrower meaning, namely, a fluid that is comprised mainly of interstitial fluid or sweat as it emerges from the skin.

As used herein, “chronological assurance” means the sampling rate or sampling interval that assures measurement(s) of analytes in a biofluid at the rate where measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30-minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

As used herein, “sweat sampling rate” is the effective rate at which new biofluid sample, originating from the pre-existing pathways, reaches a sensor that measures a property of the fluid or its solutes. Sampling rate is the rate at which new biofluid is refreshed at the one or more sensors and therefore old biofluid is removed as new fluid arrives. In one embodiment, this can be estimated based on volume, flow-rate, and time calculations, although it is recognized that some biofluid or solute mixing can occur. Sampling rate directly determines or is a contributing factor in determining the chronological assurance. Times and rates are inversely proportional (rates having at least partial units of 1/seconds), therefore a short or small time required to refill sample volume can also be said to have a fast or high sampling rate. The inverse of sampling rate (1/s) could also be interpreted as a “sampling interval(s)”. Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluid, or other measurement contamination sources for the measurement(s). Sampling rate can also be in part determined from solute generation, transport, advective transport of fluid, diffusion transport of solutes, or other factors that will impact the rate at which new sample will reach a sensor and/or is altered by older sample or solutes or other contamination sources.

As used herein, “sample generation rate” is the rate at which biofluid is generated by flow through pre-existing pathways. Sample generation rate is typically measured by the flow rate from each pre-existing pathway in nL/min/pathway. In some cases, to obtain total sample flow rate, the sample generation rate is multiplied by the number of pathways from which the sample is being sampled. Similarly, as used herein, “analyte generation rate” is the rate at which solutes move from the body or other sources toward the sensors.

As used herein, “measured” can imply an exact or precise quantitative measurement and can include broader meanings such as, for example, measuring a relative amount of change of something. Measured can also imply a binary measurement, such as ‘yes’ or ‘no’ type qualitative measurements.

As used herein, “analyte” means a substance, molecule, ion, or other material that is measured by a sweat sensing device.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence of the target analyte. Such sensors can be in the forms disclosed in U.S. Pat. Nos. 7,803,542 and 8,003,374 (the “Multiple-capture Aptamer Sensor” (MCAS)), or in U.S. Provisional Application No. 62/523,835 (the “Docked Aptamer Sensor” (DAS)).

“Analyte capture complex” means an aptamer, oligomer, or other suitable molecules or complexes, such as proteins, polymers, molecularly imprinted polymers, polypeptides, and glycans, that experience a conformation change in the presence of a target analyte, and are capable of being used in an analyte-specific sensor. Such molecules or complexes can be modified by the addition of one or more primer sections comprised of nucleotide bases.

“Aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

“Biorecognition element” means an aptamer or other molecule that interacts with a target analyte molecule and can be functionalized as part of a biosensor, including without limitation, proteins, polymers, molecularly imprinted polymers, polypeptides, and glycans.

“Aptamer sensing element” means an analyte capture complex that is functionalized to operate in conjunction with an electrode to detect the presence of a target analyte. Such functionalization may include tagging the aptamer with a redoxable moiety, or attaching thiol binding molecules, docking structures, or other components to the aptamer. Multiple aptamer sensing elements functionalized on an electrode comprise an EAB sensor.

“Sensitivity” means the change in output of the sensor per unit change in the parameter being measured. The change may be constant over the range of the sensor (linear), or it may vary (nonlinear).

“pH-independent redox moiety” means a redoxable moiety that has a redox reaction that is at least partially insensitive to changes in the H⁺ concentration of the surrounding biofluid throughout the normal pH range of the biofluid. For example, sweat pH typically ranges from 5 to 7, so a pH-independent redox moiety for use in sweat sensing would show reduced sensitivity to pH changes within this range.

### DETAILED DESCRIPTION OF THE INVENTION

Several exemplary embodiments of a pH-independent redox moiety for an EAB sensing device will now be described. The embodiments described herein may be utilized with any type of EAB sensing device that measures at least one analyte in sweat, interstitial fluid, or other biofluid. The disclosed embodiments may be applied to sensing devices which measure samples at chronologically assured sampling rates or intervals. Further, sensing devices which include a pH-independent redox moiety as described herein may take on many forms including patches, bands, straps, portions of clothing, wearables, or any other suitable mechanism that reliably brings sampling and sensing technology into intimate proximity with one or more biofluid samples as the sample is transported to the skin surface. The sensing devices may utilize adhesives or other mechanisms to hold the device secure against the skin, such as a strap, adhesive, or embedding in a helmet. Certain embodiments show sensors as simple individual elements. It is understood that many sensors require two or more electrodes, reference electrodes, or additional supporting technology or features which are not captured in the description herein. Sensors can be in duplicate, triplicate, or more, to provide improved data and reading accuracy. Certain embodiments of the disclosed invention show sub-components of what would be sensing devices with more conventional sub-components (such as a battery) needed for use of the device in various applications. For purposes of brevity and of greater focus on inventive aspects, such subcomponents are not explicitly shown or described herein.

The problem of pH variability in a biofluid sensing device is approached herein through a number of embodiments for sensing elements having an analyte capture complex tagged with a pH-independent redox moiety. The embodiments described herein provide a reagentless, pH-independent electrochemical sensor, as well as methods of using an EAB sensor to detect one or more target analytes in a variable-pH biofluid sample. The disclosed devices utilize a redoxable moiety that has a stable redox potential over the pH range of sweat, namely, pH in the range of approximately 4.5 to 7.5. In contrast to currently used redox moieties, which rely on a proton (H⁺) transfer to perform the redox reaction, the redox moieties described herein react by electron transfer, thereby reducing the effect on the redox reaction of free protons in a biofluid sample. By using a redox moiety that has a stable redox reaction over the expected pH range of a biofluid sample, the sensing element can be made less sensitive to pH changes in the sample.

Turning now to FIG. 3, which depicts a viologen compound undergoing a first and second electron transfer process. Viologen is a bipyridine having a number of analogues created by substitutions at the 4′ nitrogen, or the 2′, 3′, 5′, and 6′ carbons. In certain analogues, viologen can function as a pH-independent redox moiety by producing a detectable signal through the electron transfer reaction. The potential at which a viologen compound undergoes an electron transfer redox reaction is variable, and determined by the particular analogue. For use in a sensing element as disclosed herein, the potential for the redox reaction must be low enough to avoid current-induced dissolution of the bond(s) attaching the analyte capture complex to the electrode, which occurs at about −0.6 V, compared to the standard hydrogen brady theory electrode (“SHE”).

FIG. 4 depicts a methyl viologen analogue useable as a pH-independent redox moiety. The methyl viologen redox moiety will undergo an electron transfer reaction at −0.446 V compared to SHE at 30° C., thereby forming a resonance stabilized free-radical. This redox potential is similar to that of methylene blue. Methyl viologen will also undergo a second electron transfer reaction at around −0.75 V. This second electron transfer reaction forms a conjugated double bond system that is planar and sterically hindered at the 3′ and 5′ position by the hydrogens (or substituted moieties). The steric hindrance causes this latter compound to be less stable and more prone to oxidation. Thus, the one-electron-transfer reaction, having a highly redox stable complex, is preferable for characterizing the reaction potential of the redox moiety. The stability of the methyl viologen redox moiety can be further enhanced by bonding or linking the compound to an analyte capture complex.

A methyl viologen redox moiety may be synthesized according to the following example process, which comprises six steps that can be grouped into two stages. Steps 1 through 3 comprise the first stage, in which a linker section is prepared for attachment to a 4,4′-bipyridyl molecule.

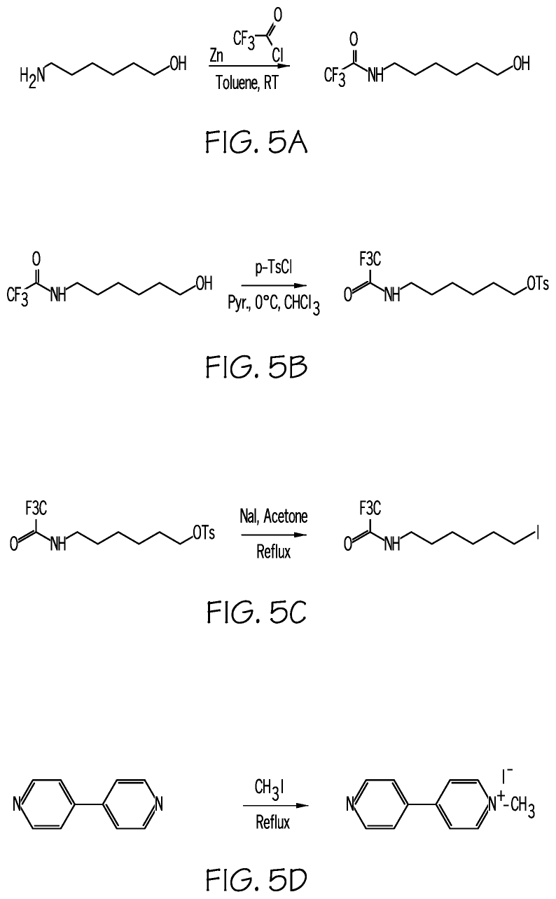

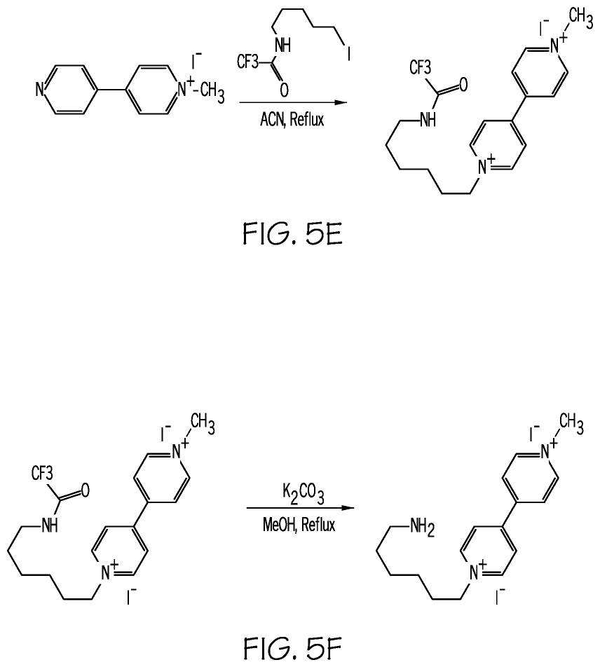

With reference to FIG. 5A, Step 1 is a zinc-mediated protection of the amide group. This step preserves the amide group's reactivity for later attachment to the analyte capture complex. The ideal yield for this step is 98%.

With reference to FIG. 5B, Step 2 is a tosylation of the alcohol group, which prepares the group for attachment to the 4,4′-bipyridyl molecule. The ideal yield for this step is 95%.

With reference to FIG. 5C, in Step 3, the linker is iodinated for attachment to the 4,4′-bipyridyl. The ideal yield for this step is 98%.

Steps 4 through 6 comprise the second stage of methyl viologen redox moiety synthesis, during which the 4,4′-bipyridyl molecule is prepared and attached to the linker section.

With reference to FIG. 5D, Step 4 is the methylation of the 4,4′-bipyridyl. The ideal yield for this step is 99%.

With reference to FIG. 5E, in Step 5, the 4,4′-bipyridyl is alkylated. The ideal yield for this step is 49%.

With reference to FIG. 5F, in Step 6, the amide is reduced to remove its protective group and to allow the methyl viologen redox moiety to be attached to the analyte capture complex. Ideal yield for this step is 100%. See Maloney, et al., Organic Letters, 2005 7 (19), 4297-4300; WIPO Publication No. WO2005062110A1; Grenier, M. C., et al., Bioorganic & Medicinal Chemistry Letters, 2012, 22 (12), 4055-4058; Meshram, H. M., et al., Tetrahedron Letters, 1998, 39 (23), 4103-4106; Ishizaki, M., et al., “Perkin Transactions 1,” J. Chem. Soc., 1993, 101-110.

In some embodiments, the methylation of the 4,4-bipyridyl (step 4) can be replaced with a variety of functional groups, including both electron-donating and electron-withdrawing groups. Other modifications of the 4,4′-bipyridyl rings may also be used to modify the chemical and physical properties of the final structure (including modifications to one or more of the 2′, 3′, 5′, or 6′ carbons). Each of these types of modifications can be used to tailor the redox moiety's properties, while maintaining an electrical potential for the stable redox reaction that is below the level that would cause dissolution of the analyte capture complex from the electrode surface. After the final structure has been synthesized, the redox moiety can be attached to the analyte capture complex either directly, via covalent bonds, or via a linking section of various lengths, including bonding an NHS group tethered to the redox moiety to the aptamer sensing element.

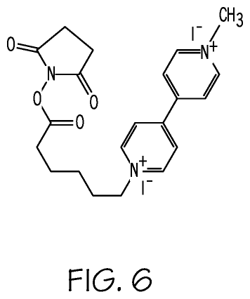

FIG. 6A depicts another embodiment of a pH-independent redox moiety, hexyl methyl viologen, which is an alternative configuration of the 4,4′-bipyridyl compound described above. The hexyl methyl viologen will undergo a first electron transfer reaction at a potential of approximately −0.5V compared to SHE at 30° C. Hexyl methyl viologen will undergo a stable second electron transfer reaction at approximately −0.7 V compared to SHE at 30° C. In this embodiment, the addition of the hexyl group to the viologen makes the viologen compound less pH-sensitive.

Hexyl methyl viologen may be synthesized according to the following example process, which comprises five steps.

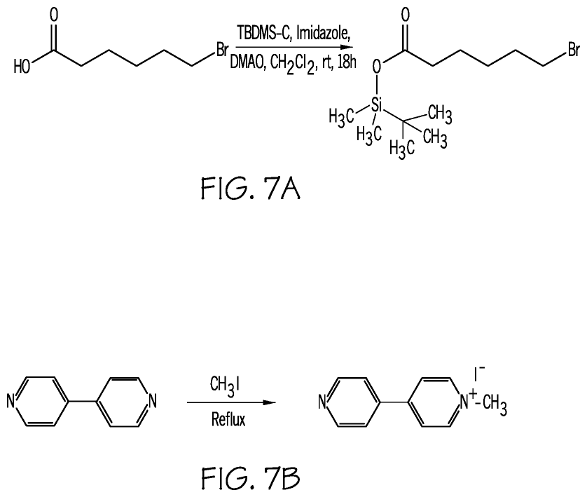

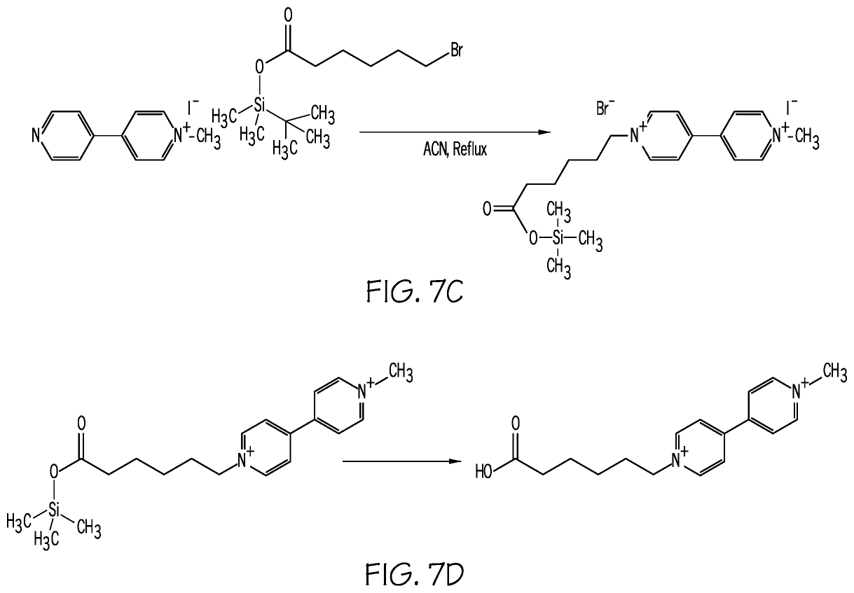

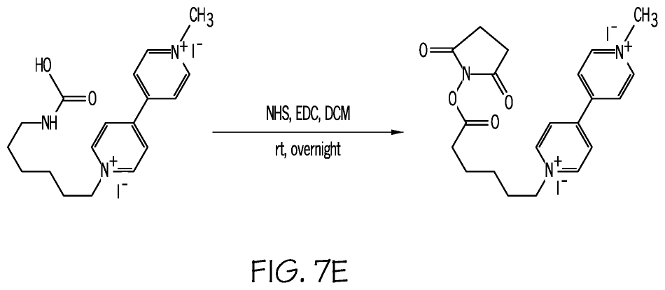

With reference to FIG. 7A, Step 1 is a protection of the linker carboxylate group. This step preserves the amide group's reactivity for later attachment to the analyte capture complex. The ideal yield for this step is 97%.

With reference to FIG. 7B, Step 2 is a methylation of the viologen. The ideal yield for this step is 99%.

With reference to FIG. 7C, in Step 3, the protected linker group is attached to the methyl viologen. The ideal yield for this step is 49%.

With reference to FIG. 7D, Step 4 is the deprotection of the linker group to allow the final product to be attached to the analyte capture complex. The ideal yield for this step is 99%.

With reference to FIG. 7E, in Step 5, the product of Step 4 is esterified to produce the final product, a hexyl methyl viologen. See Stork, G., et al., J. Am. Chem. Soc., 2001, 123, 3239-3242; Grenier, M. C., et al., Bioorg. Med. Chem., 2012, 22, 4055-4058; Yang, H., et al., Org. Lett., 2007, 9, 2993-2995; Kang, D., et al., “Survey of redox-active moieties for application in multiplexed electrochemical biosensors,” Anal. Chem., 2016, 88 (21), pp. 10452-10458.

FIG. 8 depicts an N-hydroxysuccinimide Ester of N-ferrocenylformylglycine (FcFG-NHS), which is another exemplary compound for use as a pH-independent redox moiety in an EAB sensor. In this embodiment, the primary moiety is the metal ferrocene (Fe), with the NHS reactive group allowing for conjugation of the moiety to both alpha and epsilon amino groups in peptide chains, as well as to free amino and thiol groups in nucleotides. In N-ferrocenylformylglycine, the iron center and the ring system ligands are nonreactive to acidic protons. While ferrocene has known instability issues over extended time periods, the addition of an amine group to the organo-metallic compound enables the N-ferrocenylformylglycine (FcFG) derivative of ferrocene to be more stable. Stability is also increased by extending the alkyl chain by an additional two carbon atoms. The addition of the amine group also enables the FcFG compound to have a redox potential in the desired range of 0.2 to 0.8V. Thus, the N-ferrocenylformylglycine derivative provides a stable, pH-independent redox moiety in the pH range of 4.5 to 7.5, suitable for use in an EAB sensor.

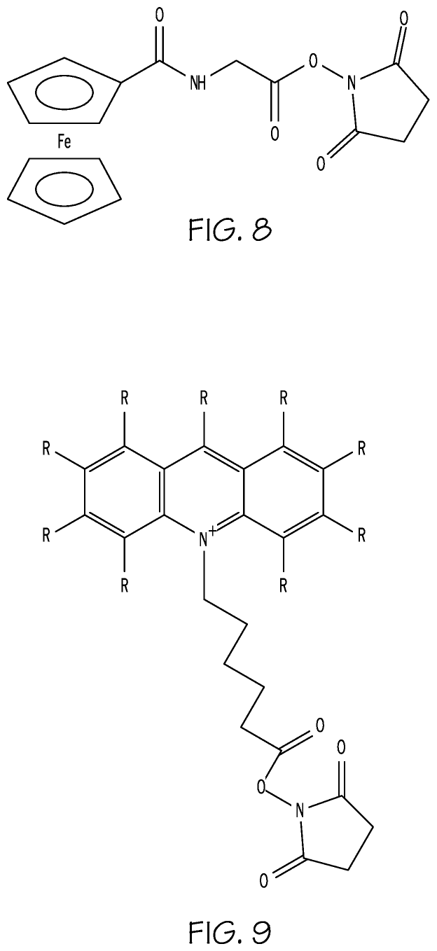

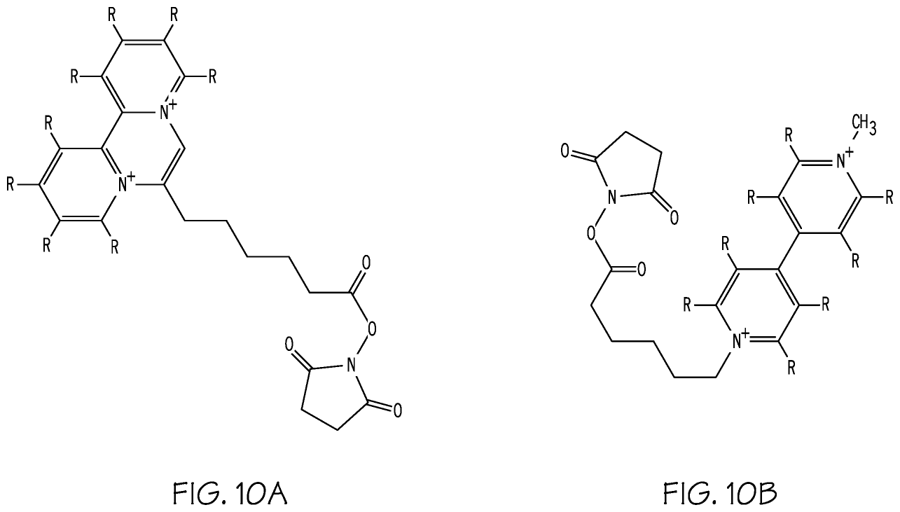

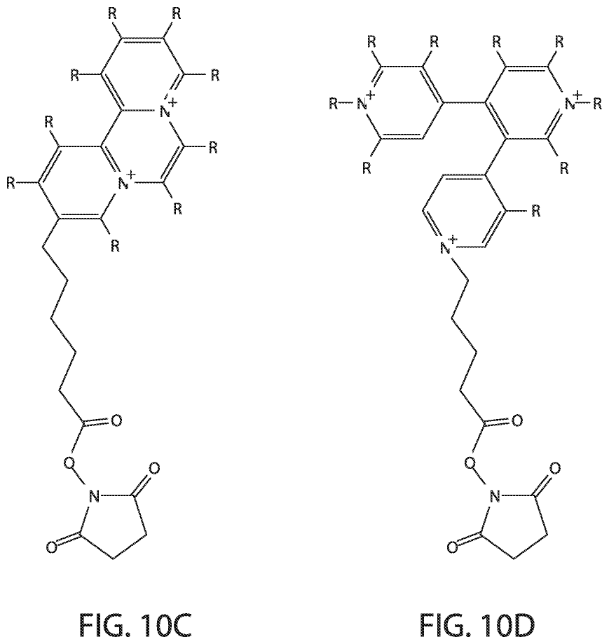

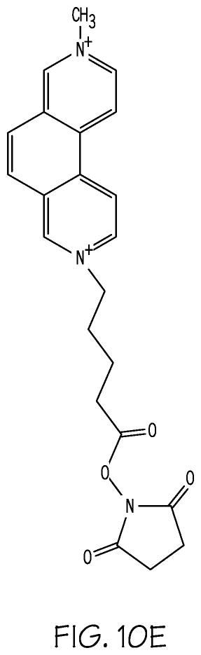

FIG. 9 depicts Hexanoyl Acridinium NHS Ester, another exemplary pH-independent redox moiety usable in an EAB sensor. In this embodiment, the pH-independent redox moiety is acridine, a multi-aromatic nitrogen containing ring system. The acridine compound can include a carbon tether to an N-hydroxysuccinimide ester, as shown in FIG. 9, for bonding the acridine compound to an analyte capture complex. The addition of an alkyl chain to the nitrogen (quaternization) in acridine eliminates the possibility of a reaction between the lone pair of electrons on the amine and acidic protons in the biofluid sample, thereby allowing the redox reaction of the acridine compound to remain stable across variations in the sample pH. FIGS. 10A through 10E depict other possible nitrogen containing ring systems related to the multi-aromatic nitrogen containing ring system of FIG. 9. These alternative compounds include different numbers and variations of nitrogen relative to the multiple aromatic rings, as well as variations in the location of the NHS tether. The acridine NHS ester is well-suited for use in an EAB biosensing device due to the stability of the compound over extended time periods.

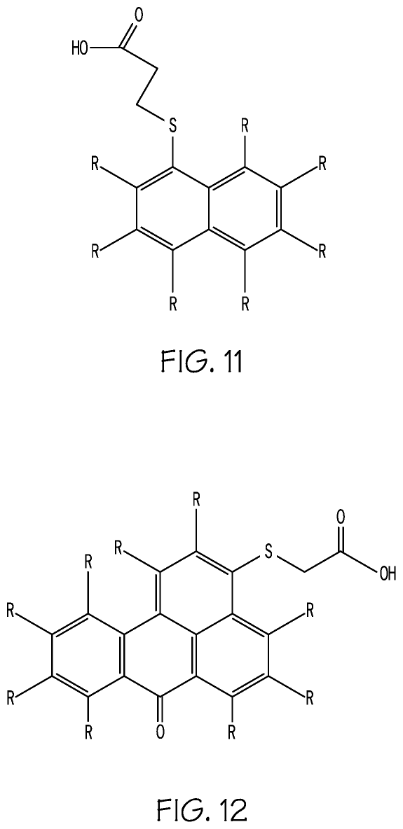

FIG. 11 depicts a 3-(1-Napthylthio)Propionic Acid, which is another exemplary pH-independent redox moiety which may be usable in an EAB sensor. In this exemplary embodiment, the main redox moiety is an aromatic sulfur-containing large ring system. A carboxylic acid is linked by thiol to the sulfur element. The carboxylic acid connected to the thiol allows for easy synthesis into an NHS ester for quick and efficient tagging of the moiety to an aptamer, utilizing free amine chemistry, to form an aptamer sensing element.

FIG. 12 depicts a 1-(7-Oxo-7H-Benz(De)Anthracen-3-ylthio) Acetic Acid, which is another exemplary pH-independent redox moiety which may be usable in an EAB sensor. In this exemplary embodiment, the main redox moiety is a large aromatic ring system including both an oxygen and a sulfur. A carboxylic acid is tethered by thiol to the sulfur. As in the previous embodiment, the tethering of thiol directly to the carboxylic acid allows for easy synthesis of the compound into an NHS ester for bonding the moiety to an aptamer of an EAB sensing element. It is anticipated that the ring systems depicted in FIGS. 11 and 12, in which a thiol is directly connected to the main moiety, will have a redox potential in the range of 0.2 to 0.8 V, which is an optimal range for use as a redox moiety in an EAB sensor.

As indicated in FIGS. 9-12 above, each of the disclosed main moieties can be bonded to one or more substituent groups “R” to tune the performance of the moiety. The substituent groups can be one or more of the functional groups chosen from a set of electron donating groups including: amines, alcohols, ethers, amides, esters, and alkyls. This set of electron donating groups is listed in the order of anticipated decreasing effect on the performance of the resulting redox moiety compound. The substituent groups “R” could also be one or more functional groups chosen from a set of electron withdrawing groups including: nitro groups, ammonium ions, sulfates, nitriles, trifluoromethyl acyl chlorides, carboxylic acids, esters, ketones, and aldehydes. These electron withdrawing groups are listed in the order of anticipated decreasing effect on the performance of the resulting redox moiety compound. Any one of the electron donating or electron withdrawing groups can be bonded to any one or more of the “R” locations, so that none, one, two, or more of the carbons are bonded to a selected one or more of the functional groups. The functional groups and bonding locations can be varied on any of the main moieties described above to adjust the redox reaction potential of the moiety to obtain a peak redox current signal within the desired potential range of 0.2 to 0.8 V. The substituent groups can be empirically analyzed at different bonding locations to select the one or more groups which allow the moiety to react at the desired redox potential.

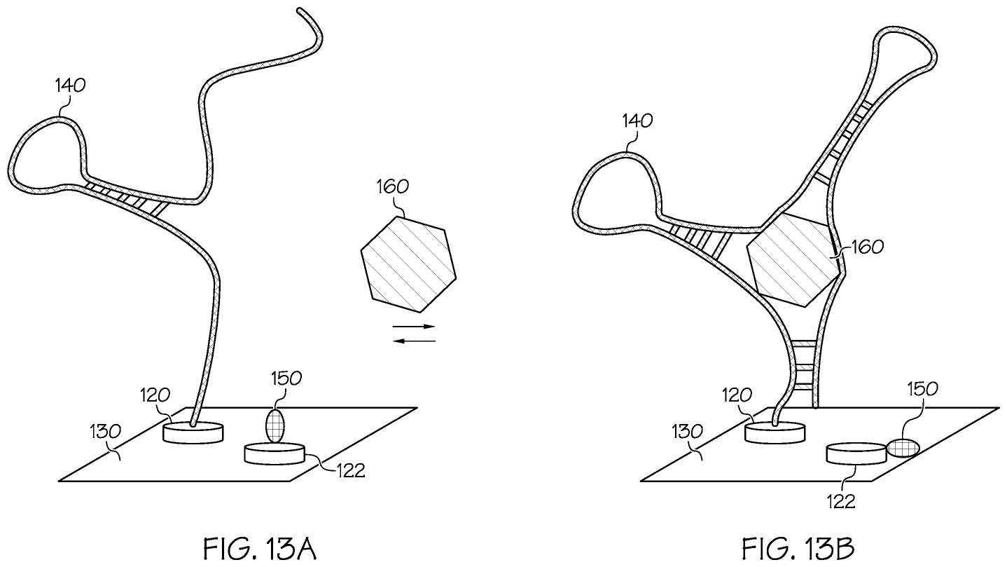

The performance of EAB sensors using the disclosed pH-independent redox moieties may be further improved for certain applications by altering the configuration of the aptamer sensing elements. For example, in some embodiments, as depicted in FIG. 13A, the redox moiety 150 may be indirectly attached (or tethered) to the electrode surface 130 via a linker (including a monothiol, a dithiol, or a trithiol) 122, and an analyte capture complex 140 may also be bound via a linker 120 to the electrode. In the presence of a target analyte 160, the analyte capture complex experiences a conformation change upon binding, as depicted in FIG. 13B. The conformation change causes the analyte capture complex 140 to directly influence the dynamics and position of the redox moiety 150, thereby altering the signal upon interrogation of the electrode 130.

In other embodiments, the performance of EAB biofluid sensing devices can be improved by employing EAB sensors configured with a plurality of different redox moieties. For example, an EAB sensor can be configured with a number of aptamer sensing elements with redox molecules that have a stable redox reaction within one pH range, and a number of aptamer sensing elements having redox molecules with stable redox reactions for an adjacent pH range. An EAB sensor can thereby be configured to include sensing elements with pH ranges sufficient to cover the expected pH range for the application. In this embodiment, a pH sensor could also be included in the device and used in concert with the EAB sensor. The pH sensor signal will allow for selection of the redox signal from the more stable redox moiety at the detected pH.

Alternatively, a biosensing device as described herein could also include an internal standard sensor constructed using a plurality of redox moieties 150 attached via linkers 122 to the surface of an electrode 130. The attachment between the redox moieties 150 and electrode 130 may be similar to that depicted in FIGS. 13A and 13B. The internal standard sensor surface would be covered with the redox moiety structures. The internal standard sensor would be next to a sensor functionalized with the aptamer sensing elements. The redox moieties on the standard sensor will respond to pH changes or other solution changes by creating a signal. The redox signal from the standard sensor can be subtracted out of the signal created from the aptamer sensing elements to account for changes in the redox moiety behavior due to pH or other solution changes.

In other embodiments, the aptamer sensing element may also include an oligonucleotide blocker section (such as a derivative of mercaptohexanol), as disclosed in U.S. Pat. No. 7,803,542, to provide performance characteristics that are desirable for certain applications. In such embodiments, the redox moiety may be attached to the blocker section, rather than the analyte capture complex. Such embodiments may also feature the redox moiety tethered to the electrode.

In addition to EAB sensors, using a pH-independent redox moiety may also improve the pH stability of other types of redox-mediated sensors. For example, electrochemical biosensors may be constructed using various analyte capture complexes employing alternate biorecognition elements, including, without limitation, proteins, polymers (especially molecularly imprinted polymers), polypeptides, glycans, and others. In a similar way to the conformational changes that characterize analyte capture for aptamer-based sensors, when such alternate biorecognition elements bind an analyte, the resulting conformation changes would produce an observable change in signal when such sensing elements are interrogated using SWV, or other suitable sensor interrogation method.

For example, some alternate sensing platforms use a peptide, an antigen, or an immobilized small molecule in the analyte capture complex. The redox moiety for such platforms may be free ferric cyanide molecules. When the analyte capture complexes bind a target analyte (such as a large protein) the target analyte sterically occludes the electrode surface so that the sensing platform produces a decreased current signal when interrogated. As disclosed herein, a pH-independent redox moiety tethered to the electrode surface can replace the ferric cyanide. When the capture molecule binds the target analyte, the pH-independent redox moiety would be forced to move relative to the electrode surface, which would change the current signal observed when the sensor element is interrogated.

Similarly, a sensing element with a tethered pH-stable redox moiety may also use a protein that is immobilized on the electrode surface as an analyte capture complex. In the absence of a target analyte, the protein, e.g., an antibody or enzyme, sterically occludes a portion of the electrode surface, which will force the redox moiety into a first position relative to the electrode surface. When interrogated by SWV, the sensing element will produce a first current output corresponding to this first relative position. Since proteins have some degree of conformational flexibility, when the immobilized protein binds to a target analyte molecule, the protein will experience a conformational change, which will cause the redox moiety to move into a second position relative to the electrode surface. When interrogated by SWV, the sensing element will accordingly produce a second current output corresponding to this second relative position.

For example, a sensing element for the detection of maltose could be fabricated by immobilizing maltose binding protein (MBP) to the electrode surface, and measuring a current signal representing the unbound state of the sensing element. In the presence of maltose, the MBP will capture some of the maltose molecules and experience conformational changes. As additional maltose binds to the MBP, the changes to MBP conformation and dynamics will result in a change in the amount of surface area sterically occluded by the MBP. This change in occluded surface area will cause the tethered pH-stable redox moiety to change its position relative to the electrode surface, which will produce an observable change in current signal when the sensing element is interrogated.

While previous work in the field has used one-electron-transfer redox reactions to form redox stable complexes, as well as substitutions to alter the redox potential of compounds, the biosensing elements and devices disclosed herein (1) use such techniques to create novel pH-independent redox moieties for biomolecule sensing, (2) pair a redox moiety with an aptamer-based sensing element, and (3) bind the redox moiety to the sensing element. The redox moiety reacts through an electron transfer in order to produce a signal change indicative of the presence of a target analyte.

Several exemplary embodiments have been described for biosensing devices having sensing elements with pH-independent redox moieties. However, it is anticipated that other structures, compounds, and configurations may also be used, provided the alternative compounds and/or configurations provide predictable redox reactions when exposed to variable pH biofluid samples. Various modifications, alterations, and adaptations to the embodiments described herein may occur to persons skilled in the art with attainment of at least some of the advantages. The disclosed embodiments are therefore intended to include all such modifications, alterations, and adaptations without departing from the scope of the embodiments as set forth herein.

1. A device, comprising:
a plurality of redox moieties, each having a reaction potential that is at least partially independent of a potential of hydrogen (pH) value of a biofluid sample;
an electrode; and
a plurality of analyte capture complexes functionalized on a surface of the electrode, wherein each complex is paired with a redox moiety, and wherein the complex undergoes a conformation change upon an interaction with a target analyte in the biofluid sample, and wherein the complex forms a first configuration relative to the electrode before the interaction and a second configuration relative to the electrode after the interaction, the conformation change producing a detectable signal change from the redox moiety upon an electronic interrogation of the electrode.

2. The device of claim 1, wherein the redox moiety undergoes an electron transfer reaction.

3. The device of claim 1, wherein the redox moiety has a reaction potential in the range of 0.2 through 0.8 volts.

4. The device of claim 1, wherein the redox moiety is selected from the group of compounds consisting of the following: a methyl viologen; a hexyl methyl viologen; an N-ferrocenylformylglycine; an acridine; a 3-(1-napthylthio)propionic acid; and a 1-(7-oxo-7H-benz(de)anthracen-3-ylthio) acetic acid.

5. The device of claim 1, wherein the redox moiety has one or more electron donating groups.

6. The device of claim 1, wherein the redox moiety has one or more electron withdrawing groups.

7. The device of claim 5, wherein the redox moiety further comprises at least one substituent selected from the group consisting of the following: an amine; an alcohol; an ether; an amide; an ester; and an alkyl.

8. The device of claim 6, wherein the redox moiety further comprises at least one substituent selected from the group consisting of the following: a nitro group; an ammonium ion; a sulfate; a nitrile; a trifluoromethyl acyl chloride; a carboxylic acid; an ester; a ketone; and an aldehyde.

9. The device of claim 1, wherein the biofluid sample primarily comprises one of the following: tears; sweat; blood; urine; interstitial fluid; serum; and plasma.

10. The device of claim 1, wherein the redox moiety has a stable reaction over a range of 4.5 pH to 7.5 pH.

11. The device of claim 1, wherein the redox moiety further includes a linker for attaching the redox moiety to one of the following: an oligonucleotide blocker; an analyte capture complex; and the electrode.

12. The device of claim 11, wherein the linker further comprises an NHS group.

13. The device of claim 1, wherein the each analyte capture complex comprises one of the following: an aptamer; a protein; an antibody; an enzyme; a peptide; a polypeptide; a polymer; a molecularly imprinted polymer; and a glycan.

14. A device, comprising:
a plurality of aptamer sensing elements, wherein each sensing element undergoes a conformational change upon an interaction with a target analyte in a biofluid sample, and wherein each sensing element forms a first configuration before the interaction and a second configuration after the interaction;
a redox moiety paired with each sensing element, the redox moiety having a reaction potential that is at least partially independent of a potential of hydrogen (pH) value of the biofluid sample; and
an electrode operative with the plurality of sensing elements, and configured to produce a first signal when interrogated in the first configuration, and a second signal when interrogated in the second configuration.

15. The device of claim 14, wherein the redox moiety undergoes an electron transfer reaction.

16. The device of claim 14, wherein the biofluid sample primarily comprises one of the following: tears; sweat; blood; urine; interstitial fluid; serum; and plasma.

17. The device of claim 14, wherein the redox moiety is selected from the group of compounds consisting of: a methyl viologen; a hexyl methyl viologen; an N-ferrocenylformylglycine; an acridine; a 3-(1-napthylthio)propionic acid; and a 1-(7-oxo-7H-benz(de)anthracen-3-ylthio) acetic acid.

18. The device of claim 14, wherein the redox moiety further comprises one or more electron donating groups.

19. The device of claim 14, wherein the redox moiety further comprises one or more electron withdrawing groups.

20. The device of claim 14, wherein the redox moiety further comprises a linker section configured to attach the redox moiety to one of the following: an oligonucleotide blocker; an aptamer sensing element; and the electrode.

21. The device of claim 20, wherein the linker section is an NHS group.

22. A device, comprising:
a plurality of analyte capture complexes, each configured to undergo a conformational change upon an interaction with a target analyte in a biofluid sample, each complex forming a first configuration before the interaction and a second configuration after the interaction;
a pH-insensitive redox moiety paired with each complex, the pH-insensitive redox moiety reacting through an electron transfer; and
an electrode operative with the plurality of complexes, and configured to produce a first signal when interrogated in the first configuration, and a second signal when interrogated in the second configuration.
